(19) United States
(12) Patent Application Publication
(10) Pub. No.: US 2024/0249827 A1
(43) Pub. Date: Jul. 25, 2024

(54) IMAGE PROCESSING DEVICE, IMAGE PROCESSING METHOD, AND RECORDING MEDIUM

(71) Applicant: SONY GROUP CORPORATION, TOKYO (JP)

(72) Inventors: SHO INAYOSHI, TOKYO (JP); SOTA SHOMAN, TOKYO (JP); SATOSHI OZAKI, TOKYO (JP)

(21) Appl. No.: 18/559,415

(22) PCT Filed: Feb. 10, 2022
(86) PCT No.: PCT/JP2022/005246

§ 371 Date: Nov. 7, 2023

(30) Foreign Application Priority Data
May 26, 2021 (JP) 2021-088335

(51) Int. Cl. G16H 30/40; G06V 10/77; G16H 30/20

(52) U.S. Cl. CPC G16H 30/40; G06V 10/7715; G16H 30/20

(57) ABSTRACT

An image processing device (40) of a form according to the present disclosure includes: a feature amount extraction section (42a) that extracts an intermediate feature amount related to machine learning from an input image that is an image inside a body; an importance calculation section (42b) that calculates image importance of the input image on the basis of the intermediate feature amount; and an image accumulation section (42c) that stores the input image on the basis of the image importance.

### FIELD

The present disclosure relates to an image processing device, an image processing method, and a recording medium.

### BACKGROUND

In a surgical environment such as a laparoscopic endoscope, a situation in which a recognizer that is learned by machine learning and that recognizes a surgical tool or the like assists surgery is assumed. In general, recognition performance of a recognizer learned by machine learning tends to deteriorate due to a difference between a learning environment and an inference environment, such as a difference in an illumination condition or a used surgical tool. Under the inference environment, it is not possible to determine whether an image currently being photographed is useful data for learning of the recognizer (importance). Thus, it is difficult to efficiently obtain data contributing to improvement of the recognition performance. On the other hand, in the machine learning, a technology of prioritizing labeling of when active learning is performed by using usefulness for learning of unlabeled data has been proposed (see, for example, Patent Literature 1).

### CITATION LIST

### Patent Literature

Patent Literature 1: Japanese Patent Application Laid-open No. 2020-154602

### SUMMARY

### Technical Problem

However, in the above-described technology, importance of data (image) cannot be obtained in real time, and it is difficult to efficiently obtain data contributing to improvement of the recognition performance. For example, in the above-described technology, it is assumed that an output of a learning model is reliable, and additional labeling is necessary. Thus, it is difficult to efficiently obtain the data contributing to the improvement of the recognition performance.

Thus, the present disclosure proposes an image processing device, an image processing method, and a recording medium capable of efficiently obtaining data contributing to improvement of recognition performance.

### Solution to Problem

An image processing device according to the embodiment of the present disclosure includes: a feature amount extraction section that extracts an intermediate feature amount related to machine learning from an input image that is an image inside a body; an importance calculation section that calculates image importance of the input image on a basis of the intermediate feature amount; and an image accumulation section that stores the input image on a basis of the image importance.

An image processing method according to the embodiment of the present disclosure includes: extracting an intermediate feature amount related to machine learning from an input image that is an image inside a body; calculating image importance of the input image on a basis of the intermediate feature amount; and storing the input image on a basis of the image importance.

A computer-readable recording medium, according to the embodiment of the present disclosure, recording a program for causing a computer to execute: extracting an intermediate feature amount related to machine learning from an input image that is an image inside a body; calculating image importance of the input image on a basis of the intermediate feature amount; and storing the input image on a basis of the image importance.

### BRIEF DESCRIPTION OF DRAWINGS

FIG. 1 is a diagram illustrating an example of a schematic configuration of an image processing system according to an embodiment.

FIG. 2 is a diagram for describing an example of preliminary learning processing according to the embodiment.

FIG. 3 is a diagram for describing an example of inference processing according to the embodiment.

FIG. 4 is a flowchart illustrating an example of a flow of learning processing according to the embodiment.

FIG. 5 is a flowchart illustrating an example of a flow of inference processing according to the embodiment.

FIG. 6 is a diagram for describing an example of comparison of individual intermediate feature amounts of learned data and an input image according to the embodiment.

FIG. 7 is a diagram for describing an example of a display of an image according to the embodiment.

FIG. 8 is a first diagram for describing an example of learning model application processing according to the embodiment.

FIG. 9 is a second diagram for describing an example of learning model application processing according to the embodiment.

FIG. 10 is a diagram illustrating an example of a schematic configuration of a computer.

FIG. 11 is a diagram illustrating an example of a schematic configuration of an endoscope system.

FIG. 12 is a block diagram illustrating an example of a functional configuration of a camera and a camera control unit (CCU) illustrated in FIG. 11.

FIG. 13 is a diagram illustrating an example of a schematic configuration of a microscopic surgery system.

### DESCRIPTION OF EMBODIMENTS

In the following, embodiments of the present disclosure will be described in detail on the basis of the drawings. Note that a device, a system, a method, a recording medium, and the like according to the present disclosure are not limited by these embodiments. In addition, the same reference signs are basically assigned to components having substantially the same functional configuration, and overlapped description is omitted in the present specification and the drawings.

Each of one or a plurality of embodiments (including examples and modification examples) described in the following can be performed independently. On the other hand, at least a part of the plurality of embodiments described in the following may be appropriately combined with at least a part of the other embodiments. The plurality of embodiments may include novel features different from each other. Thus, the plurality of embodiments can contribute to solving different objects or problems, and can exhibit different effects.

The present disclosure will be described in the following order of items.
1. Embodiment
1.1. Configuration example of an image processing system
1-2. Example of learning processing
1-3. Example of inference processing
1-4. Comparative example of individual intermediate feature
amounts of learned data and an input image
1-5. Display example of an image
1-6. Example of learning model application processing
1-7. Action/effect
2. Other embodiments
3. Configuration example of a computer
4. Application example
5. Supplementary note

### 1. Embodiment

### <1.1 Configuration Example of an Image Processing System>

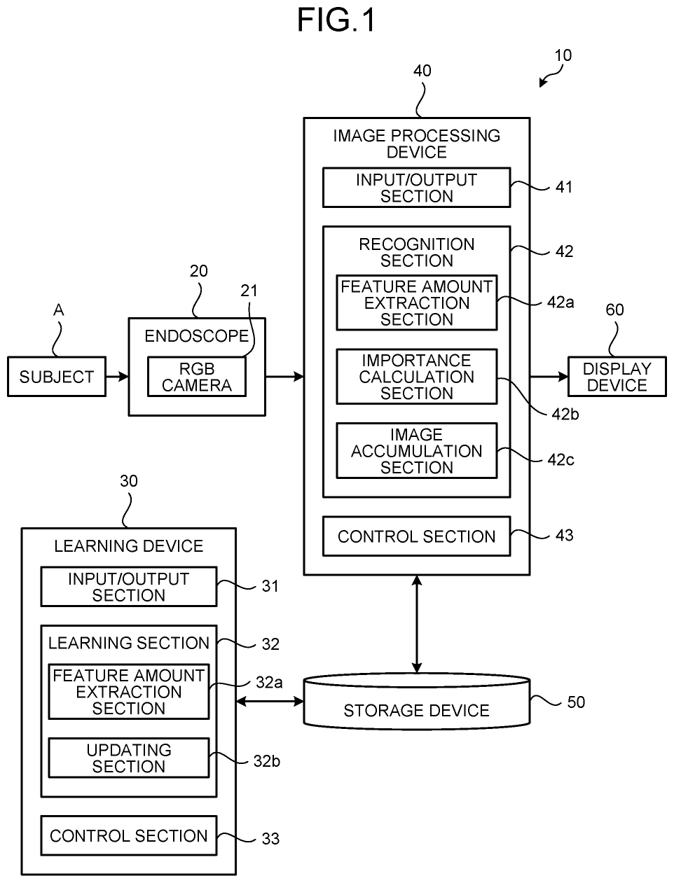

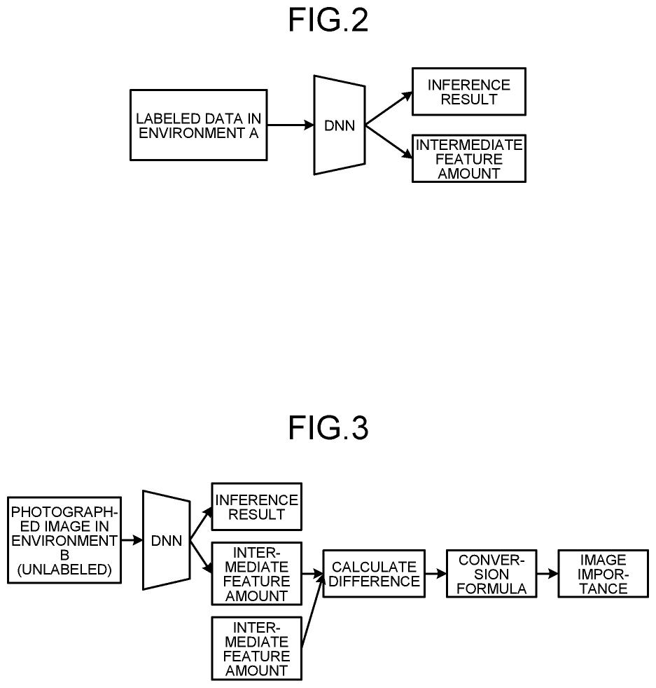

A configuration example of an image processing system 10 according to the present embodiment will be described with reference to FIG. 1 to FIG. 3. FIG. 1 is a diagram illustrating an example of a schematic configuration of the image processing system 10 according to the present embodiment. FIG. 2 is a diagram for describing an example of preliminary learning processing according to the present embodiment. FIG. 3 is a diagram for describing an example of inference processing according to the present embodiment.

As illustrated in FIG. 1, the image processing system 10 includes an endoscope 20, a learning device 30, an image processing device 40, a storage device 50, and a display device 60. This image processing system 10 is a system that processes an image (such as an image inside a body) of a subject A such as a patient.

### (Endoscope)

The endoscope 20 includes an RGB camera 21. The RGB camera 21 mainly includes, for example, a plurality of pixels arrayed in a matrix, and a peripheral circuit section that outputs an image, which is based on light incident on each of the plurality of pixels, as a pixel signal (both are not illustrated). This RGB camera 21 functions as an imaging section that photographs a photographing target in a body of the subject A in a form of a moving image or a still image. For example, the RGB camera 21 can obtain an image of an intraperitoneal environment of the subject A (for example, an operative field image including various surgical tools and organs in an abdominal cavity). Furthermore, the RGB camera 21 transmits the captured image (such as a pixel signal corresponding to an image) to the image processing device 40.

Specifically, the RGB camera 21 is an image sensor capable of color photographing, and is, for example, an image sensor having a Bayer array capable of detecting blue light, green light, and red light. Furthermore, the RGB camera 21 is preferably, for example, an image sensor applicable to photographing of a high resolution image of 4K or more. By using such an image sensor, an image of a surgical site can be obtained with high resolution. Thus, an operator such as a surgeon can grasp a state of the surgical site in more detail and can smoothly proceed with the operation.

Note that the endoscope 20 may be, for example, an oblique viewing scope, a front direct viewing scope with a wide angle/cutout function, an endoscope with a distal end bending function, or an endoscope with another direction simultaneous photographing function, or may be a flexible endoscope or a rigid endoscope, and is not specifically limited. Furthermore, the RGB camera 21 may include a pair of image sensors for respectively acquiring right-eye and left-eye images corresponding to 3D display (stereoscopic system). In a case of performing the 3D display, the operator such as the surgeon can more accurately grasp a depth of a body tissue (organ) in the surgical site and can grasp a distance to the body tissue.

### (Learning Device)

The learning device 30 includes an input/output section 31, a learning section 32, and a control section 33.

The input/output section 31 receives labeled data (image data) for preliminary learning and data (image data) in the storage device 50 and inputs the data to the learning section 32. In addition, the input/output section 31 outputs various kinds of data related to learning by the learning section 32 to the storage device 50.

The learning section 32 performs preliminary learning by machine learning such as a deep neural network (DNN) with labeled data for preliminary learning, constructs a learned model, and stores the learned model together with an intermediate feature amount and the like in the storage device 50 via the input/output section 31.

For example, as illustrated in FIG. 2, at the time of preliminary learning, the learning section 32 inputs labeled data (image data) in an environment A to the DNN, obtains an inference result and an intermediate feature amount, and stores the obtained inference result and intermediate feature amount in the storage device 50. In the obtaining of the inference result, for example, learning is performed by back propagation of an error from a correct answer label. In the obtaining of the intermediate feature amount, for example, after the learning is completed, the intermediate feature amount for each piece of data (intermediate feature amount for each image) is stored. Note that in each piece of data, an average or variance (such as an average vector), a representative value, or the like of the intermediate feature amounts may be stored. Furthermore, examples of problem setting of inference include detection of a surgical tool in an image, segmentation of an organ, and the like.

Returning back to FIG. 1, the learning section 32 includes a feature amount extraction section 32a and an updating section 32b. The feature amount extraction section 32a extracts an intermediate feature amount of image data such as labeled data and unlabeled data. The updating section 32b updates the learned model and intermediate feature amount stored in the storage device 50 according to a difference in image obtaining environments (such as the environment A, environment B, and the like). Examples of a difference in environment include a difference in hospitals, a difference in operating rooms, and the like. For example, lighting conditions, surgical tools, and the like vary depending on a hospital or an operating room.

The control section 33 controls each section (such as the input/output section 31, the learning section 32, or the like) of the learning device 30. For example, the control section 33 includes a computer such as a central processing unit (CPU) or a micro processing unit (MPU), and can integrally control the operation of each section of the learning device 30.

### (Image Processing Device)

The image processing device 40 includes an input/output section 41, a recognition section 42, and a control section 43.

The input/output section 41 receives image data (pixel signal) from the endoscope 20 and inputs the image data to the recognition section 42, outputs various kinds of data related to recognition by the recognition section 42 to the storage device 50, and outputs the image data and the like to the display device 60.

The recognition section 42 performs learning by machine learning such as the DNN on the unlabeled data (image data), obtains an estimation result and an intermediate feature amount, and performs storing thereof into the storage device 50 via the input/output section 41. These estimation result, intermediate feature amount, and the like are used for additional learning such as domain adaptive learning.

For example, as illustrated in FIG. 3, the recognition section 42 inputs unlabeled data (image data) in the environment B to the DNN, obtains an estimation result and an intermediate feature amount, and stores the obtained estimation result and intermediate feature amount in the storage device 50. In addition, the recognition section 42 calculates a difference between the obtained intermediate feature amount of the unlabeled data in the environment B and the intermediate feature amount of the labeled data in the environment A, converts the difference between the intermediate feature amounts by a predetermined conversion formula, and obtains image importance. The conversion formula is, for example, image importance=difference/constant. Various functions such as this function can be applied as the conversion formula. Note that a method of obtaining the inference result and the intermediate feature amount, problem setting of inference, and the like are the same as those of the learning section 32 of the learning device 30 described above.

Returning back to FIG. 1, the recognition section 42 includes a feature amount extraction section 42a, an importance calculation section 42b, and an image accumulation section 42c. The feature amount extraction section 42a extracts the intermediate feature amount from the image data such as the unlabeled data (such as an input image that is an image inside a body). The importance calculation section 42b calculates image importance of an image (such as an RGB image) on the basis of the intermediate feature amount of the image data. The image accumulation section 42c stores the image in the storage device 50 on the basis of the image importance. For example, the image accumulation section 42c stores and accumulates the image with high image importance.

The control section 43 controls each section (such as the input/output section 41, the recognition section 42, or the like) in the image processing device 40. For example, the control section 43 includes a computer such as a CPU or an MPU, and can integrally control operation of each section of the image processing device 40. Furthermore, the control section 43 controls the endoscope 20, the display device 60, and the like. For example, the control section 43 can transmit a control signal to each of the endoscope 20 and the display device 60 and control driving thereof. The control signal for the endoscope 20 may include information related to imaging conditions such as magnification and a focal length.

### (Storage Device)

The storage device 50 stores various kinds of data such as an image captured by the RGB camera 21 (such as the RGB image or the like), the learned model, the intermediate feature amount for each image, and the like. The storage device 50 is realized by, for example, a storage device such as a hard disk drive (HDD) or a solid state drive (SDD).

### (Display Device)

The display device 60 displays various images such as the image obtained by the RGB camera 21. The display device 60 is realized by, for example, a display including a liquid crystal display (LCD), an organic electro-luminescence (EL) display, or the like. Note that the display device 60 may be a device integrated with the image processing device 40, or may be a separate device communicably connected to the image processing device 40 in a wired or wireless manner.

### <1-2. Example of Learning Processing>

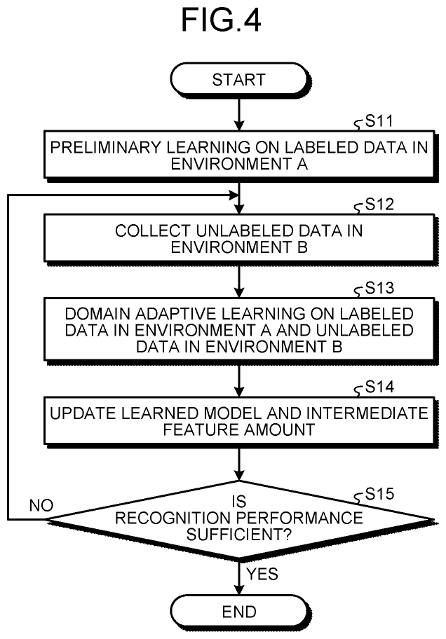

An example of learning processing according to the present embodiment will be described with reference to FIG. 4. FIG. 4 is a flowchart illustrating an example of a flow of the learning processing according to the present embodiment. The learning processing is executed by the learning device 30. For example, the learning section 32 executes the learning processing.

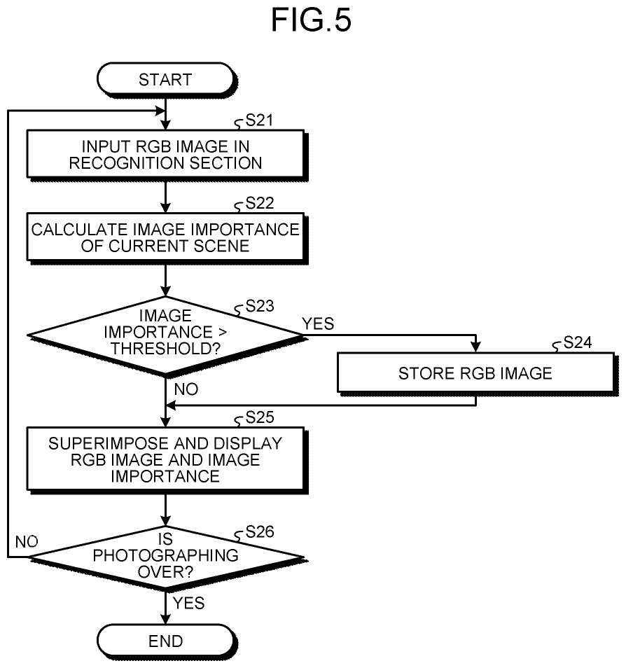

As illustrated in FIG. 4, preliminary learning is executed in Step S11 on the labeled data in the environment A. The unlabeled data in the environment B is collected in Step S12. The unlabeled data is stored in the storage device 50 by inference processing (described later) (see FIG. 5), and is read from the storage device 50 and used. In Step S13, domain adaptive learning is executed on the labeled data in the environment A and the unlabeled data in the environment B. In Step S14, the learned model and the intermediate feature amount are updated according to the domain adaptive learning.

Then, in Step S15, it is determined whether the recognition performance is sufficient. When it is determined that the recognition performance is not sufficient (No in Step S15), the processing returns to Step S12, and Step S12 to S15 are repeated. On the other hand, when it is determined that the recognition performance is sufficient (Yes in Step S15), the processing ends. Whether the recognition performance is sufficient may be determined, for example, by a user, or may be automatically determined by the learning device 30. Note that in the determination by the user, for example, an input section such as a keyboard, a mouse, or a touch panel is operated by the user, and it is input that the recognition performance is sufficient or insufficient. In the determination by the learning device 30, for example, the recognition performance is quantified, and it is determined that the recognition performance is sufficient or insufficient depending on whether the numerical value is larger than a threshold.

### <1-3. Example of Inference Processing>

An example of the inference processing according to the present embodiment will be described with reference to FIG. 5. FIG. 5 is a flowchart illustrating an example of a flow of the inference processing according to the present embodiment. The inference processing is executed by the image processing device 40. For example, the recognition section 42 executes the inference processing.

As illustrated in FIG. 5, the RGB image (input image) is input to the recognition section 42 in Step S21. In Step S22, image importance of a current scene (input image) is calculated. In Step S23, it is determined whether the image importance is higher than a predetermined threshold. When it is determined that the image importance is higher than the threshold (Yes in Step S23), the RGB image is stored in the storage device 50 in Step S24, and the RGB image and the image importance are superimposed and displayed by the display device 60 in Step S25. On the other hand, when it is determined that the image importance is not higher than the threshold (No in Step S23), the RGB image and the image importance are superimposed and displayed by the display device 60 directly in Step S25. Note that in Step S24, for example, the RGB image and the image importance may be stored in the storage device 50 in association with each other.

Then, it is determined in Step S26 whether photographing is over. When it is determined that the photographing is not over (No in Step S26), the processing returns to Step S21, and Step S21 to S26 are repeated. On the other hand, when it is determined that the photographing is over (Yes in Step S26), the processing is ended. For example, the user determines whether the photographing is over. In this determination by the user, similarly to the above, for example, the input section such as the keyboard, the mouse, or the touch panel is operated by the user, and it is input that the photographing is over or not over.

### <1-4. Comparative Example of Individual Intermediate Feature Amounts of Learned Data and an Input Image>

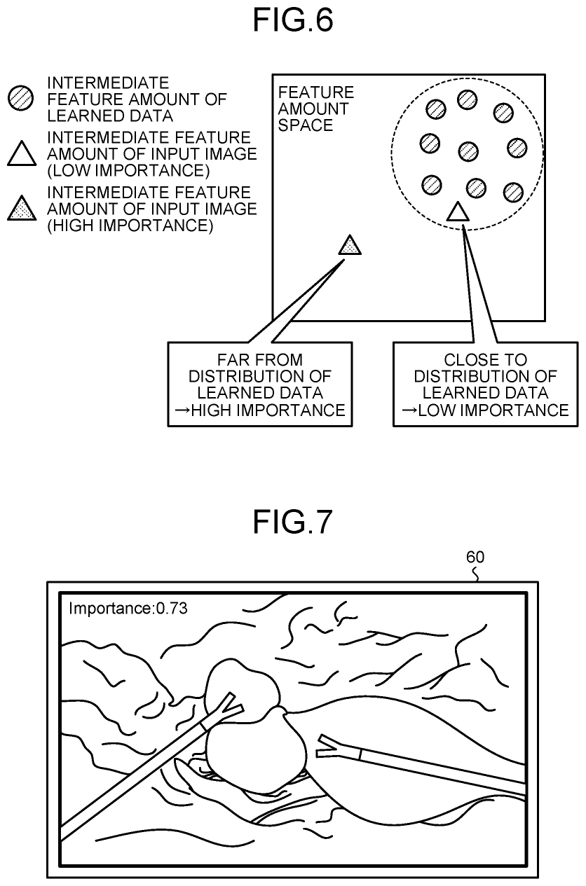

A comparative example of the individual intermediate feature amounts of the learned data and the input image according to the present embodiment will be described with reference to FIG. 6. FIG. 6 is a diagram for describing an example of comparison of individual intermediate feature amounts of the learned data and the input image according to the present embodiment.

As illustrated in FIG. 6, by comparing the intermediate feature amount of the learned data with the intermediate feature amount of the input image, it is possible to determine whether an image currently being photographed is data necessary for the additional learning. That is, in a case where the intermediate feature amount of the input image is close to a distribution of the intermediate feature amount of the learned data (see a dotted line region in FIG. 6), the image importance of the input image is determined to be low. In the example of FIG. 6, the intermediate feature amount of the input image having the low image importance is located in the dotted line region. On the other hand, in a case where the intermediate feature amount of the input image is far from the distribution of the intermediate feature amount of the learned data (see the dotted line region in FIG. 6), it is determined that the image importance of the input image is high.

Specifically, such image importance is a normalized value calculated from a difference between the intermediate feature amounts of the learned data and the input image. The importance is lower as the image importance is closer to 0, and the importance is higher as the image importance is closer to 1.

However, it is assumed that a value of the image importance becomes smaller each time the learning model is repeatedly updated. For example, in the example of FIG. 6, the intermediate feature amount of the learned data increases each time the update of the learning model is repeated, and the distribution region of the intermediate feature amount of the learned data (see the dotted line region in FIG. 6) becomes wider. Thus, the intermediate feature amount of the input image becomes closer to the distribution of the intermediate feature amount of the learned data, and the value of the image importance tends to become smaller (the image importance tends to become lower). Since it is assumed that the value of the image importance becomes smaller each time the learning model is repeatedly updated in such a manner, for example, it is preferable to update the threshold (see Step S23 in FIG. 5) each time the learning model is updated.

The threshold of the image importance can be changed, and may be changed by the user or may be automatically changed by the image processing device 40, for example. In the change by the user, similarly to the above, the input section such as the keyboard, the mouse, or the touch panel is operated by the user, and the threshold is changed, for example. In the change by the image processing device 40, the threshold may be periodically changed, or the threshold may be changed according to update timing or the number of times of update of the learning model. For example, when the number of times of update becomes a predetermined number of times, the threshold is changed. This change processing is executed by the image accumulation section 42c, for example.

### <1-5. Display Example of an Image>

A display example of an image according to the present embodiment will be described with reference to FIG. 7. FIG. 7 is a diagram for describing an example of a display of an image according to the present embodiment.

As illustrated in FIG. 7, the currently-captured RGB image (input image) and the image importance are superimposed and displayed by the display device 60 (superimposed display). As described above, the importance is lower as the image importance is closer to 0, and the importance is higher as the image importance is closer to 1. In the example of FIG. 7, the image importance of the RGB image is 0.73 (importance: 0.73). Since this image importance is displayed, the user can grasp the image importance.

Furthermore, in a case where the image importance is higher than the threshold, a color of an outer frame (see a thick black frame in FIG. 7) of the RGB image is changed. As a result, the user can grasp that the image importance is higher than the threshold. For example, the color of the outer frame is blue in a case where the image importance is equal to or lower than the threshold, and the color of the outer frame is changed to red in a case where the image importance is higher than the threshold. In such a manner, a display mode of the image indicating the outer frame is changed according to the image importance. However, the color (combination of colors) of the outer frame is not limited to blue and red, and other colors may be used.

Note that the display mode of the image indicating the outer frame may be changed by, for example, blinking of the outer frame or changing of a thickness (line width) or a size of the outer frame in addition to changing of the color of the outer frame. That is, the color, line width, and size of the image indicating that the image importance is higher than the threshold may be changed, or the image may be blinked. In addition, as the image indicating that the image importance is higher than the threshold, an image indicating a character, a symbol, a figure, or the like may be used in addition to the outer frame. In addition, both or one of the image indicating that the image importance is higher than the threshold and the image importance may or may not be superimposed on the RGB image.

As described above, since the image importance of the currently-captured image data is displayed, a user (photographer) such as an operator or an assistant can grasp the image importance of the currently-captured image data, and can efficiently photograph image data that contributes to improvement of the recognition performance. For example, the user such as the operator or the assistant intensively captures a scene in a case where the image importance of the currently-captured image data is high, and captures a different scene in a case where the image importance of the currently-captured image data is low. In such a manner, it is possible to supplement imaging left to discretion of the user and cause the user to continue imaging.

### <1-6. Example of Learning Model Application Processing>

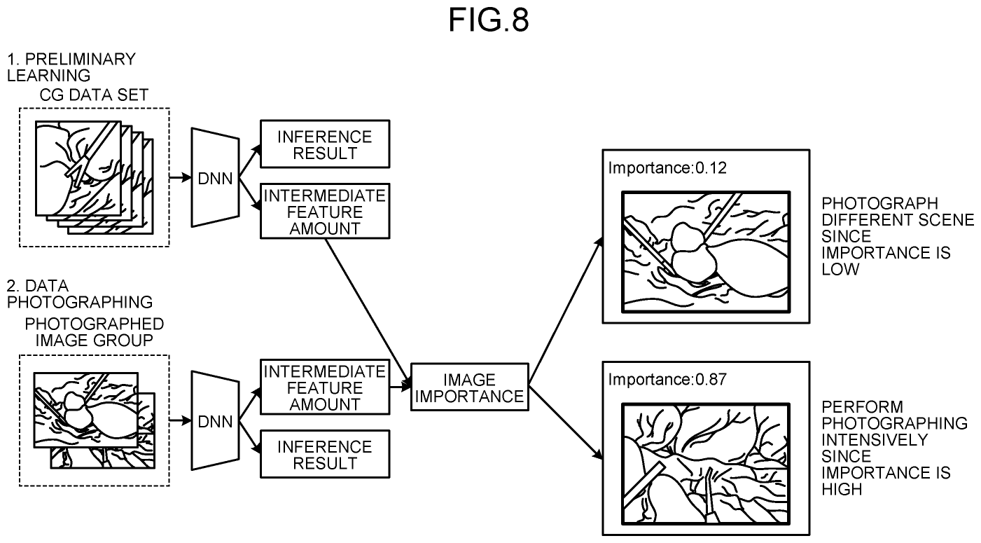

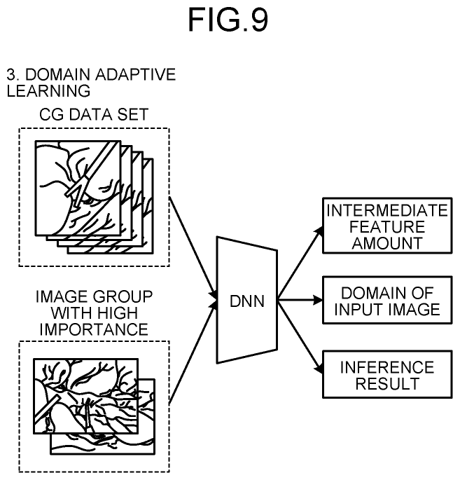

An example of the learning model application processing with respect to the environment according to the present embodiment will be described with reference to FIG. 8 and FIG. 9. FIG. 8 and FIG. 9 are respectively diagrams for describing examples of the learning model application processing according to the present embodiment.

As illustrated in FIG. 8, in 1. Preliminary learning, the learning section 32 of the learning device 30 inputs a CG data set to the DNN, obtains an inference result and an intermediate feature amount, and stores the obtained inference result and intermediate feature amount in the storage device 50. The learning model is constructed by the preliminary learning. In the example of FIG. 8, a CG data set is used as labeled data for preliminary learning. This CG data set is a data set including a plurality of images (images during surgery) generated by computer graphics (CG).

In 2. Data photographing, the recognition section 42 of the image processing device 40 inputs a photographed image (captured image) group (image data) to the DNN, obtains an estimation result and an intermediate feature amount, and stores the obtained estimation result and intermediate feature amount in the storage device 50. Furthermore, the recognition section 42 calculates, for each photographed image, a difference between the intermediate feature amount of the photographed image and the intermediate feature amount of the image included in the CG data set (such as an average value, a representative value, or the like of each piece of data), converts a difference between the intermediate feature amounts by a conversion formula, and obtains the image importance. The recognition section 42 generates the photographed image including the image importance by superimposing the obtained image importance on the photographed image, and transmits the generated image as a display image to the display device 60.

The display device 60 displays the display image transmitted from the recognition section 42. In the example of FIG. 8, since image importance of an upper display image is 0.12 (importance: 0.12) and the image importance is equal to or lower than the threshold (such as 0.50), the color of the outer frame is blue. In addition, since image importance of a lower display image is 0.87 (importance: 0.87) and the image importance is higher than the threshold (such as 0.50), the color of the outer frame is red. Note that the image importance is not superimposed on the photographed image, and is indicated above the photographed image (in FIG. 8) while the photographed image being avoided.

The user (photographer) such as the operator or the assistant can visually recognize the display image displayed by the display device 60 and grasp the image importance. The user sees the image importance, and takes a measure such as intensively photographing the current scene, or stopping photographing the current scene and moving to photographing of a different scene. In such a manner, the user can grasp the image importance of the currently-captured image data, and efficiently photograph the image data having the high image importance. As a result, image data contributing to improvement in the recognition performance is sequentially captured and accumulated, whereby the image data contributing to the improvement in the recognition performance can be efficiently obtained. Furthermore, by changing the color of the outer frame of the image according to whether the image importance is higher or lower than the threshold, it is possible to make it easier for the user to understand whether the image importance is high or low. For example, the outer frame of the image is set to red and indicates an alert in a case where the image importance is high, and the outer frame of the image is set to blue in a case where the image importance is low. As a result, the user can easily grasp a degree of the image importance.

As illustrated in FIG. 9, in 3. Domain adaptive learning, the learning section 32 of the learning device 30 inputs the CG data set and an image group having the image importance higher than the threshold to the DNN, obtains an inference result, an intermediate feature amount, and a domain of an input image, and stores the obtained inference result, intermediate feature amount, and domain of the input image in the storage device 50. As a specific example, the learning section 32 updates the learned model (learned DNN model) and the saved intermediate feature amount.

Note that in the obtaining of the inference result, for example, only labeled data is subjected to back propagation of an error from the correct answer label and performance of learning. Furthermore, in the determination of the domain of the input image, learning is performed in such a manner that the DNN makes a wrong determination of the domain of the input image (adversarial learning). In the obtaining of the intermediate feature amount, for example, after the learning is completed, the intermediate feature amount for each piece of data is stored.

Such 2. Data photographing and 3. Domain adaptive learning are repeated until sufficient recognition performance is obtained. Note that although the images having the image importance exceeding the threshold are accumulated and the domain adaptive learning is performed in a case where labeling is not performed, this is not a limitation. For example, labeling (labeling) may be performed and supervised learning may be performed.

Furthermore, in 1. Preliminary learning, the CG data set is used. As a result, automatic labeling can be performed, and a large amount of labeled data is obtained at low cost. By adapting this learning model to an environment of each hospital by the domain adaptive learning using the image importance, it is possible to obtain the learning model with the high recognition performance in the environment of each hospital without performing costly labeling. Note that, usually, it is necessary to photograph and label a large amount of data for each environment (hospital) in which the learning model is introduced, which is unrealistic in consideration of cost. However, according to the processing of 1 to 3, the learning model with the high recognition performance in the environment of each hospital can be obtained at low cost.

In such learning model application processing, labeling is performed on data photographed in a certain environment, the DNN is learned in advance, and the intermediate feature amount that is an intermediate output of the DNN for each image is stored. An intermediate feature amount is calculated by utilization of the DNN at the time of photographing of data in an introduction environment, a difference from the stored intermediate feature amount is calculated, the difference is calculated as image importance of the data, and feedback thereof is given to a data photographer. Note that the present embodiment is different from a learning model, in which additional labeling is assumed, since the additional labeling is not assumed. Furthermore, in the present embodiment, data necessary for the additional learning is collected with a focus on the intermediate feature amount instead of an output of the DNN learning model.

In such a manner, by the real-time feedback of the image importance of the currently photographed image during the photographing, data can be efficiently collected even at the time of data photographing. For example, under the inference environment, whether the scene (image) currently being photographed is useful data for learning of the recognition section 42 (image importance) is output in real time. As a result, it becomes possible to efficiently image data having the high image importance and sequentially store data contributing to improvement in the recognition performance of the recognition section 42, whereby it is possible to efficiently obtain the data contributing to the improvement in the recognition performance. As an example, as described above, by performing the domain adaptive learning by using the labeled data and the unlabeled data having the high image importance, it is possible to improve the recognition performance of the recognition section 42 without performing costly labeling.

### <1-7. Action/Effect>

As described above, according to the embodiment, the feature amount extraction section 42a that extracts the intermediate feature amount related to the machine learning from the input image that is the image in the body, the importance calculation section 42b that calculates the image importance of the input image on the basis of the intermediate feature amount, and the image accumulation section 42c that stores the input image on the basis of the image importance are provided. As a result, the input image can be stored according to the image importance of the input image, and the input image contributing to the improvement in the recognition performance can be reliably stored, whereby the input image (data) contributing to the improvement in the recognition performance can be efficiently obtained.

Furthermore, the importance calculation section 42b may calculate the image importance on the basis of a difference between the intermediate feature amount of the image inside the body in a first environment (such as the hospital A) and the intermediate feature amount of the input image in a second environment different from the first environment (such as the hospital B). As a result, the image importance related to the environment can be reliably calculated.

Furthermore, the importance calculation section 42b may calculate the image importance by converting the difference by the predetermined conversion formula. As a result, the image importance related to the environment can be reliably calculated by easy processing.

In addition, the first environment may be a first hospital, and the second environment may be a second hospital different from the first hospital. As a result, it is possible to calculate the image importance related to the hospital as the environment.

Furthermore, in a case where the image importance exceeds the predetermined threshold, the image accumulation section 42c may accumulate the input image. As a result, the input image can be reliably stored by easy processing according to the image importance.

In addition, the image accumulation section 42c may change the predetermined threshold at the update timing of the learned model. As a result, since the threshold is changed at appropriate timing at which the learned model is updated, the input image can be stored according to the image importance even when the learned model is repeatedly updated.

In addition, the image accumulation section 42c may change the predetermined threshold according to the number of times of update of the learned model. As a result, since the threshold is changed according to the number of times of update of the learned model, the input image can be stored according to the image importance even when the learned model is repeatedly updated.

In addition, the image accumulation section 42c may reduce the predetermined threshold at timing at which the number of times of update becomes a predetermined number of times. As a result, the threshold is changed to be small when the number of times the learned model is updated becomes the predetermined number of times, whereby the input image can be stored according to the image importance even when the learned model is repeatedly updated.

Furthermore, the image accumulation section 42c may store the input image and the image importance in association with each other. As a result, the input image and the image importance can be read and used, whereby convenience thereof as data can be improved.

In addition, the display device 60 that displays image importance is provided. As a result, the user can grasp the image importance of the input image, efficiently photograph the image with the high image importance, and store the input image contributing to the improvement in the recognition performance, whereby the input image (data) contributing to the improvement in the recognition performance can be efficiently obtained.

In addition, the display device 60 may display the input image and the image importance. As a result, since being able to visually recognize the input image and the image importance, the user can easily grasp the image importance corresponding to the input image.

In addition, the display device 60 may display the input image with the image importance being superimposed thereon. As a result, it becomes easy for the user to visually recognize the image importance while visually recognizing the input image. Thus, it is possible to reliably grasp the image importance corresponding to the input image.

In addition, the display device 60 may display the image (such as an image indicating an outer frame, a character, a symbol, a figure, or the like) indicating that the image importance exceeds the predetermined threshold. As a result, since being able to visually recognize the image indicating that the image importance exceeds the predetermined threshold, the user can easily grasp that the image importance exceeds the predetermined threshold.

In addition, according to the image importance, the display device 60 may change the display mode of the image indicating that the image importance exceeds the predetermined threshold. As a result, the user can easily and reliably grasp that the image importance is changed.

In addition, the display device 60 may display the image indicating that the image importance exceeds the predetermined threshold in such a manner as to be superimposed on the input image. As a result, since being able to visually recognize the image indicating that the image importance exceeds the predetermined threshold while visually recognizing the input image, the user can reliably grasp that the image importance exceeds the predetermined threshold.

In addition, the display device 60 may display the input image, the image importance, and the image indicating that the image importance exceeds the predetermined threshold. As a result, since being able to visually recognize the input image, the image importance, and the image indicating that the image importance exceeds the predetermined threshold, the user can easily grasp the image importance corresponding to the input image and grasp that the image importance exceeds the predetermined threshold.

In addition, the display device 60 may display the image importance and the image indicating that the image importance exceeds the predetermined threshold in such a manner as to be superimposed on the input image. This makes it easy for the user to visually recognize the image importance and the image indicating that the image importance exceeds the predetermined threshold while visually recognizing the input image, whereby the user can reliably grasp the image importance corresponding to the input image and grasp that the image importance exceeds the predetermined threshold.

### 2. Other Embodiments

The processing according to the above-described embodiments (or modification examples) may be performed in various different forms (modification examples) other than the above-described embodiment. For example, among the pieces of processing described in the above embodiments, a whole or part of the processing described to be automatically performed can be manually performed, or a whole or part of the processing described to be manually performed can be automatically performed by a known method. In addition, the processing procedures, specific names, and information including various kinds of data or parameters described in the above document or in the drawings can be arbitrarily changed unless otherwise specified. For example, various kinds of information illustrated in each of the drawings are not limited to the illustrated information.

Also, each component of each of the illustrated devices is a functional concept, and does not need to be physically configured in the illustrated manner. That is, a specific form of distribution/integration of each device is not limited to what is illustrated in the drawings, and a whole or part thereof can be functionally or physically distributed/integrated in an arbitrary unit according to various loads and usage conditions.

Also, the above-described embodiments (or modification examples) can be arbitrarily combined in a range in which the processing contents do not contradict with each other. Also, the effect described in the present specification is merely an example and is not a limitation, and there may be another effect.

In addition, in the above-described embodiments (or modification examples), a system means a set of a plurality of components (such as devices, or modules (parts)), and it does not matter whether all the components are in the same housing. Thus, a plurality of devices housed in separate housings and connected via a network, and one device in which a plurality of modules is housed in one housing are both systems.

Furthermore, in the embodiments (or modification examples), a configuration of cloud computing in which one function is shared and processed in cooperation by a plurality of devices via a network can be adopted. Furthermore, each step described in the above-described processing flow (such as the flowchart) can be executed by one device, or can be shared and executed by a plurality of devices. Furthermore, in a case where a plurality of pieces of processing is included in one step, the plurality of pieces of processing included in the one step can be executed by one device or can be shared and executed by a plurality of devices.

Furthermore, the endoscope 20 may be a stereoscopic endoscope that can perform ranging. Alternatively, the endoscope 20 may include a depth sensor (ranging device) separately from the RGB camera 21. The depth sensor is, for example, a sensor that performs ranging by using a time of flight (ToF) method in which ranging is performed by utilization of a return time of reflection of pulsed light from a subject, or a structured light method in which lattice-shaped pattern light is emitted and ranging is performed according to distortion of the pattern.

### 3. Configuration Example of a Computer

The above-described series of processing can be executed by hardware or can be executed by software. In a case where the series of processing is executed by software, a program included in the software is installed in a computer. Here, examples of the computer include a computer incorporated in dedicated hardware, a general-purpose personal computer capable of executing various functions by installation of various programs, and the like.

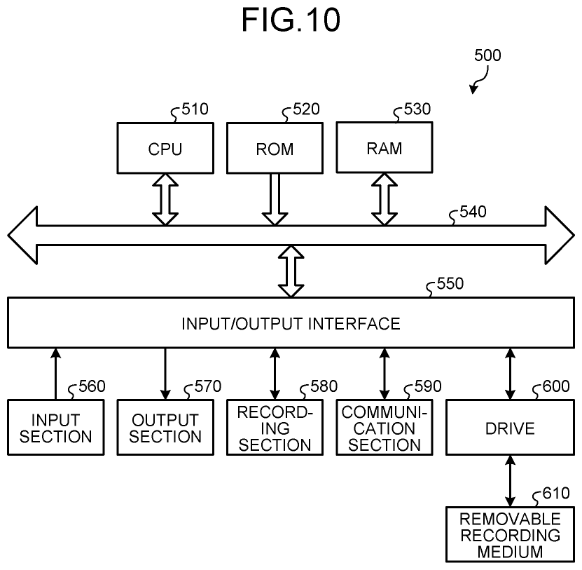

FIG. 10 is a diagram illustrating an example of a schematic configuration of a computer 500 that executes the above-described series of processing by a program.

As illustrated in FIG. 10, the computer 500 includes a central processing unit (CPU) 510, a read only memory (ROM) 520, and a random access memory (RAM) 530.

The CPU 510, the ROM 520, and the RAM 530 are connected to each other by a bus 540. An input/output interface 550 is further connected to the bus 540. An input section 560, an output section 570, a recording section 580, a communication section 590, and a drive 600 are connected to the input/output interface 550.

The input section 560 includes a keyboard, a mouse, a microphone, an imaging sensor, and the like. The output section 570 includes a display, a speaker, and the like. The recording section 580 includes a hard disk, a nonvolatile memory, and the like. The communication section 590 includes a network interface and the like. The drive 600 drives a removable recording medium 610 such as a magnetic disk, an optical disk, a magneto-optical disk, or a semiconductor memory.

In the computer 500 configured in the above manner, for example, the CPU 510 loads a program recorded in the recording section 580 into the RAM 530 via the input/output interface 550 and the bus 540 and executes the program, whereby the above-described series of processing is performed.

The program executed by the computer 500, that is, the CPU 510 can be provided by being recorded in the removable recording medium 610 as a package medium or the like, for example. Furthermore, the program can be provided via a wired or wireless transmission medium such as a local area network, the Internet, or digital satellite broadcasting.

In the computer 500, the program can be installed in the recording section 580 via the input/output interface 550 by attachment of the removable recording medium 610 to the drive 600. Furthermore, via a wired or wireless transmission medium, the program can be received by the communication section 590 and installed in the recording section 580. In addition, the program can be installed in the ROM 520 or the recording section 580 in advance.

Note that the program executed by the computer 500 may be a program in which processing is performed in time series in order described in the present specification, or may be a program in which processing is performed in parallel or at necessary timing such as when a call is made.

### 4. Application Example

The technology according to the present disclosure can be applied to a medical imaging system. The medical imaging system is a medical system using an imaging technology, and is, for example, an endoscope system or a microscope system. In the image processing system 10 according to the present disclosure, the endoscope 20 can be applied to an endoscope 5001 and a microscope device 5301, the learning device 30, the image processing device 40, and the like can be applied to a CCU 5039, the storage device 50 can be applied to a recording device 5053, and the display device 60 can be applied to a display device 5041.

### [Endoscope System]

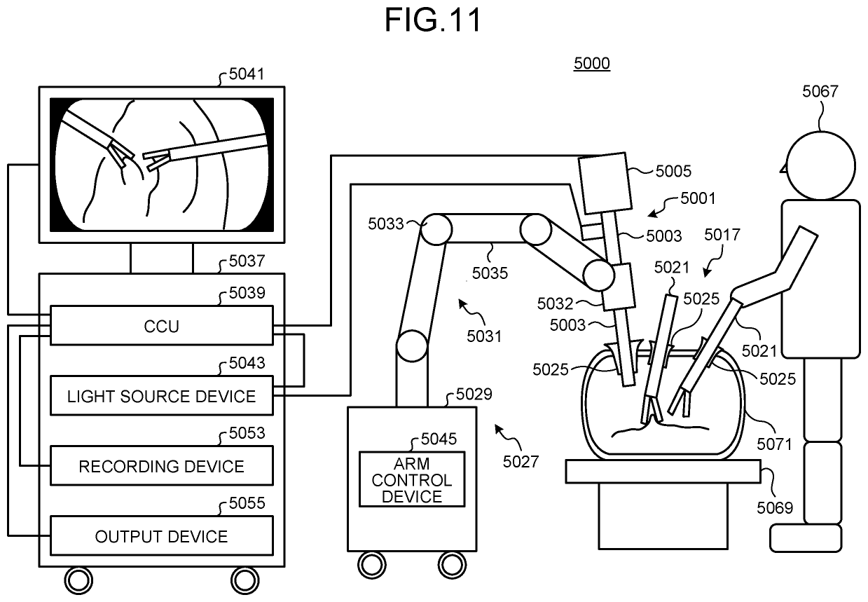

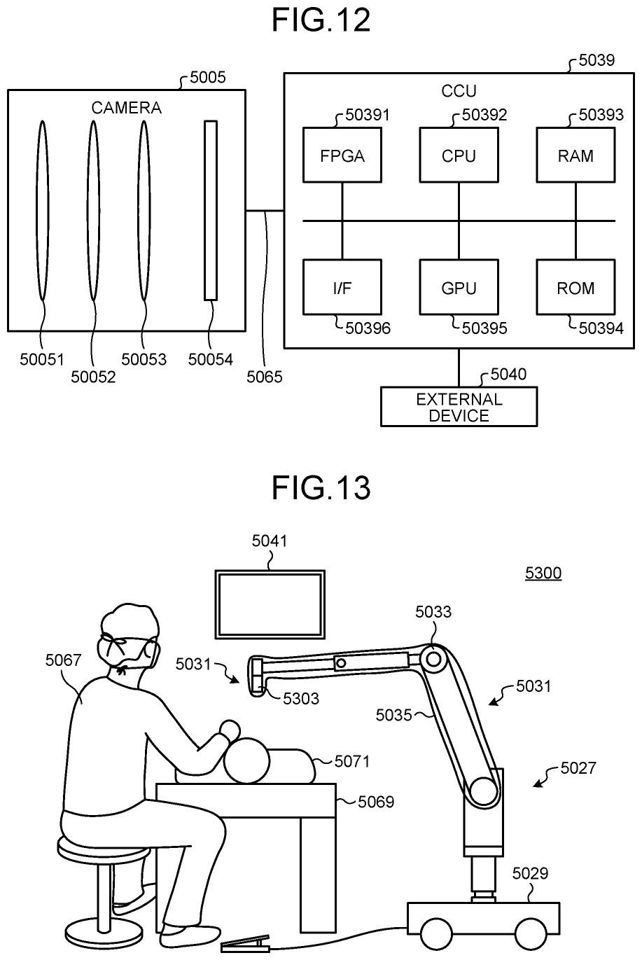

An example of the endoscope system will be described using FIGS. 11 and 12. FIG. 11 is a diagram illustrating an example of a schematic configuration of an endoscope system 5000 to which the technology according to the present disclosure is applicable. FIG. 12 is a diagram illustrating an example of a configuration of an endoscope 5001 and a camera control unit (CCU) 5039. FIG. 11 illustrates a situation where an operator (for example, a doctor) 5067 who is a participant of an operation performs the operation on a patient 5071 on a patient bed 5069 using the endoscope system 5000. As illustrated in FIG. 11, the endoscope system 5000 includes the endoscope 5001 that is a medical imaging device, the CCU 5039, a light source device 5043, a recording device 5053, an output device 5055, and a support device 5027 for supporting the endoscope 5001.

In endoscopic surgery, insertion assisting tools called trocars 5025 are punctured into the patient 5071. Then, a scope 5003 connected to the endoscope 5001 and surgical tools 5021 are inserted into a body of the patient 5071 through the trocars 5025. The surgical tools 5021 include: an energy device such as an electric scalpel; and forceps, for example.

A surgical image that is a medical image in which the inside of the body of the patient 5071 is captured by the endoscope 5001 is displayed on a display device 5041. The operator 5067 performs a procedure on a surgical target using the surgical tools 5021 while viewing the surgical image displayed on the display device 5041. The medical image is not limited to the surgical image, and may be a diagnostic image captured during diagnosis.

### [Endoscope]

The endoscope 5001 is an imaging section for capturing the inside of the body of the patient 5071, and is, for example, as illustrated in FIG. 12, a camera including a condensing optical system 50051 for condensing incident light, a zooming optical system 50052 capable of optical zooming by changing a focal length of the imaging section, a focusing optical system 50053 capable of focus adjustment by changing the focal length of the imaging section, and a light receiving sensor 50054. The endoscope 5001 condenses the light through the connected scope 5003 on the light receiving sensor 50054 to generate a pixel signal, and outputs the pixel signal through a transmission system to the CCU 5039. The scope 5003 is an insertion part that includes an objective lens at a distal end and guides the light from the connected light source device 5043 into the body of the patient 5071. The scope 5003 is, for example, a rigid scope for a rigid endoscope and a flexible scope for a flexible endoscope. The scope 5003 may be a direct viewing scope or an oblique viewing scope. The pixel signal only needs to be a signal based on a signal output from a pixel, and is, for example, a raw signal or an image signal. The transmission system connecting the endoscope 5001 to the CCU 5039 may include a memory, and the memory may store parameters related to the endoscope 5001 and the CCU 5039. The memory may be disposed at a connection portion of the transmission system or on a cable. For example, the memory of the transmission system may store the parameters before shipment of the endoscope 5001 or the parameters changed when current is applied, and an operation of the endoscope may be changed based on the parameters read from the memory. A set of the camera and the transmission system may be referred to as an endoscope. The light receiving sensor 50054 is a sensor for converting the received light into the pixel signal, and is, for example, a complementary metal-oxide-semiconductor (CMOS) imaging sensor. The light receiving sensor 50054 is preferably an imaging sensor having a Bayer array capable of color imaging. The light receiving sensor 50054 is also preferably an imaging sensor having a number of pixels corresponding to a resolution of, for example, 4K (3840 horizontal pixels×2160 vertical pixels), 8K (7680 horizontal pixels×4320 vertical pixels), or square 4K (3840 or more horizontal pixels×3840 or more vertical pixels). The light receiving sensor 50054 may be one sensor chip, or a plurality of sensor chips. For example, a prism may be provided to separate the incident light into predetermined wavelength bands, and the wavelength bands may be imaged by different light receiving sensors. A plurality of light receiving sensors may be provided for stereoscopic viewing. The light receiving sensor 50054 may be a sensor having a chip structure including an arithmetic processing circuit for image processing, or may be a sensor for time of flight (ToF). The transmission system is, for example, an optical fiber cable system or a wireless transmission system. The wireless transmission only needs to be capable of transmitting the pixel signal generated by the endoscope 5001, and, for example, the endoscope 5001 may be wirelessly connected to the CCU 5039, or the endoscope 5001 may be connected to the CCU 5039 via a base station in an operating room. At this time, the endoscope 5001 may transmit not only the pixel signal, but also simultaneously information (for example, a processing priority of the pixel signal and/or a synchronization signal) related to the pixel signal. In the endoscope, the scope may be integrated with the camera, and the light receiving sensor may be provided at the distal end of the scope.

### [Camera Control Unit (CCU)]

The CCU 5039 is a control device for controlling the endoscope 5001 and the light source device 5043 connected to the CCU 5039 in an integrated manner, and is, for example, as illustrated in FIG. 12, an image processing device including a field-programmable gate array (FPGA) 50391, a central processing unit (CPU) 50392, a random access memory 50393, a read-only memory (ROM) 50394, a graphics processing unit (GPU) 50395, and an interface (I/F) 50396. The CCU 5039 may control the display device 5041, the recording device 5053, and the output device 5055 connected to the CCU 5039 in an integrated manner. The CCU 5039 controls, for example, irradiation timing, irradiation intensity, and a type of an irradiation light source of the light source device 5043. The CCU 5039 also performs image processing, such as development processing (for example, demosaic processing) and correction processing, on the pixel signal output from the endoscope 5001, and outputs the processed image signal (for example, an image) to an external device such as the display device 5041. The CCU 5039 also transmits a control signal to the endoscope 5001 to control driving of the endoscope 5001. The control signal is information on an imaging condition such as a magnification or the focal length of the imaging section. The CCU 5039 may have a function to down-convert the image, and may be configured to be capable of simultaneously outputting a higher-resolution (for example, 4K) image to the display device 5041 and a lower-resolution (for example, high-definition (HD)) image to the recording device 5053.

The CCU 5039 may be connected to external equipment (such as a recording device, a display device, an output device, and a support device) via an IP converter for converting the signal into a predetermined communication protocol (such as the Internet Protocol (IP)). The connection between the IP converter and the external equipment may be established using a wired network, or a part or the whole of the network may be established using a wireless network. For example, the IP converter on the CCU 5039 side may have a wireless communication function, and may transmit the received image to an IP switcher or an output side IP converter via a wireless communication network, such as the fifth-generation mobile communication system (5G) or the sixth-generation mobile communication system (6G).

### [Light Source Device]

The light source device 5043 is a device capable of emitting the light having predetermined wavelength bands, and includes, for example, a plurality of light sources and a light source optical system for guiding the light of the light sources. The light sources are, for example, xenon lamps, light-emitting diode (LED) light sources, or laser diode (LD) light sources. The light source device 5043 includes, for example, the LED light sources corresponding to three respective primary colors of red (R), green (G), and blue (B), and controls output intensity and output timing of each of the light sources to emit white light. The light source device 5043 may include a light source capable of emitting special light used for special light observation, in addition to the light sources for emitting normal light for normal light observation. The special light is light having a predetermined wavelength band different from that of the normal light being light for the normal light observation, and is, for example, near-infrared light (light having a wavelength of 760 nm or longer), infrared light, blue light, or ultraviolet light. The normal light is, for example, the white light or green light. In narrow band imaging that is a kind of special light observation, blue light and green light are alternately emitted, and thus the narrow band imaging can image a predetermined tissue such as a blood vessel in a mucosal surface at high contrast using wavelength dependence of light absorption in the tissue of the body. In fluorescence observation that is a kind of special light observation, excitation light is emitted for exciting an agent injected into the tissue of the body, and fluorescence emitted by the tissue of the body or the agent as a label is received to obtain a fluorescent image, and thus the fluorescence observation can facilitate the operator to view, for example, the tissue of the body that is difficult to be viewed by the operator with the normal light. For example, in fluorescence observation using the infrared light, the infrared light having an excitation wavelength band is emitted to an agent, such as indocyanine green (ICG), injected into the tissue of the body, and the fluorescence light from the agent is received, whereby the fluorescence observation can facilitate viewing of a structure and an affected part of the tissue of the body. In the fluorescence observation, an agent (such as 5-aminolevulinic acid (5-ALA)) may be used that emits fluorescence in a red wavelength band by being excited by the special light in a blue wavelength band. The type of the irradiation light of the light source device 5043 is set by control of the CCU 5039. The CCU 5039 may have a mode of controlling the light source device 5043 and the endoscope 5001 to alternately perform the normal light observation and the special light observation. At this time, information based on a pixel signal obtained by the special light observation is preferably superimposed on a pixel signal obtained by the normal light observation. The special light observation may be an infrared light observation to observe a site inside the surface of an organ and a multi-spectrum observation utilizing hyperspectral spectroscopy. A photodynamic therapy may be incorporated.

### [Recording Device]

The recording device 5053 is a device for recording the pixel signal (for example, an image) acquired from the CCU 5039, and is, for example, a recorder. The recording device 5053 records an image acquired from the CCU 5039 in a hard disk drive (HDD), a Super Density Disc (SDD), and/or an optical disc. The recording device 5053 may be connected to a network in a hospital to be accessible from equipment outside the operating room. The recording device 5053 may have a down-convert function or an up-convert function.

### [Display Device]

The display device 5041 is a device capable of displaying the image, and is, for example, a display monitor. The display device 5041 displays a display image based on the pixel signal acquired from the CCU 5039. The display device 5041 may include a camera and a microphone to function as an input device that allows instruction input through gaze recognition, voice recognition, and gesture.

### [Output Device]

The output device 5055 is a device for outputting the information acquired from the CCU 5039, and is, for example, a printer. The output device 5055 prints, for example, a print image based on the pixel signal acquired from the CCU 5039 on a sheet of paper.

### [Support Device]

The support device 5027 is an articulated arm including a base 5029 including an arm control device 5045, an arm 5031 extending from the base 5029, and a holding part 5032 mounted at a distal end of the arm 5031. The arm control device 5045 includes a processor such as a CPU, and operates according to a predetermined computer program to control driving of the arm 5031. The support device 5027 uses the arm control device 5045 to control parameters including, for example, lengths of links 5035 constituting the arm 5031 and rotation angles and torque of joints 5033 so as to control, for example, the position and attitude of the endoscope 5001 held by the holding part 5032. This control can change the position or attitude of the endoscope 5001 to a desired position or attitude, makes it possible to insert the scope 5003 into the patient 5071, and can change the observed area in the body. The support device 5027 functions as an endoscope support arm for supporting the endoscope 5001 during the operation. Thus, the support device 5027 can play a role of a scopist who is an assistant holding the endoscope 5001. The support device 5027 may be a device for holding a microscope device 5301 to be described later, and can be called a medical support arm. The support device 5027 may be controlled using an autonomous control method by the arm control device 5045, or may be controlled using a control method in which the arm control device 5045 performs the control based on input of a user. The control method may be, for example, a master-slave method in which the support device 5027 serving as a slave device (replica device) that is a patient cart is controlled based on a movement of a master device (primary device) that is an operator console at a hand of the user. The support device 5027 may be remotely controllable from outside the operating room.

The example of the endoscope system 5000 to which the technology according to the present disclosure is applicable has been described above. For example, the technology according to the present disclosure may be applied to a microscope system.

### [Microscope System]

FIG. 13 is a diagram illustrating an example of a schematic configuration of a microscopic surgery system to which the technology according to the present disclosure is applicable. In the following description, the same components as those of the endoscope system 5000 will be denoted by the same reference numerals, and the description thereof will not be repeated.

FIG. 13 schematically illustrates a situation where the operator 5067 performs an operation on the patient 5071 on the patient bed 5069 using a microscopic surgery system 5300. For the sake of simplicity, FIG. 13 does not illustrate a cart 5037 among the components of the microscopic surgery system 5300, and illustrates the microscope device 5301 instead of the endoscope 5001 in a simplified manner. The microscope device 5301 may refer to a microscope 5303 provided at the distal end of the links 5035, or may refer to the overall configuration including the microscope 5303 and the support device 5027.

As illustrated in FIG. 13, during the operation, the microscopic surgery system 5300 is used to display an image of a surgical site captured by the microscope device 5301 in a magnified manner on the display device 5041 installed in the operating room. The display device 5041 is installed in a position facing the operator 5067, and the operator 5067 performs various procedures, such as excision of an affected part, on the surgical site while observing the state of the surgical site using the image displayed on the display device 5041. The microscopic surgery system is used in, for example, ophthalmic operation and neurosurgical operation.

The respective examples of the endoscope system 5000 and the microscopic surgery system 5300 to which the technology according to the present disclosure is applicable have been described above. Systems to which the technology according to the present disclosure is applicable are not limited to such examples. For example, the support device 5027 can support, at the distal end thereof, another observation device or another surgical tool instead of the endoscope 5001 or the microscope 5303. Examples of the other applicable observation device include forceps, tweezers, a pneumoperitoneum tube for pneumoperitoneum, and an energy treatment tool for incising a tissue or sealing a blood vessel by cauterization. By using the support device to support the observation device or the surgical tool described above, the position thereof can be more stably fixed and the load of the medical staff can be lower than in a case where the medical staff manually supports the observation device or the surgical tool. The technology according to the present disclosure may be applied to a support device for supporting such a component other than the microscope.

The technology according to the present disclosure can be suitably applied to the endoscope 5001, the microscope device 5301, the CCU 5039, the display device 5041, the light source device 5043, and the like among the above-described configurations. Specifically, operation and processing according to each embodiment can be executed in the endoscope system 5000, the microscopic surgery system 5300, and the like. By applying the technology according to the present disclosure to the endoscope system 5000, the microscopic surgery system 5300, and the like, data contributing to improvement in recognition performance can be efficiently obtained.

### 5. Supplementary Note

Note that the present technology can also have the following configurations.
(1)
An image processing device comprising:
a feature amount extraction section that extracts an intermediate feature amount related to machine learning from an input image that is an image inside a body;
an importance calculation section that calculates image importance of the input image on a basis of the intermediate feature amount; and
an image accumulation section that stores the input image on a basis of the image importance.
(2)
The image processing device according to (1), wherein
the importance calculation section calculates the image importance on a basis of a difference between the intermediate feature amount of the image inside the body in a first environment and the intermediate feature amount of the input image in a second environment different from the first environment.
(3)
The image processing device according to (2), wherein
the importance calculation section converts the difference by a predetermined conversion formula and calculates the image importance.
(4)
The image processing device according to (2) or (3), wherein
the first environment is a first hospital, and
the second environment is a second hospital different from the first hospital.
(5)
The image processing device according to any one of (1) to (4), wherein
the image accumulation section stores the input image in a case where the image importance exceeds a predetermined threshold.
(6)
The image processing device according to (5), wherein
the image accumulation section changes the predetermined threshold at update timing of a learned model.
(7)
The image processing device according to (5), wherein
the image accumulation section changes the predetermined threshold according to a number of times of update of a learned model.
(8)
The image processing device according to (7), wherein
the image accumulation section decreases the predetermined threshold at timing at which the number of times of update becomes a predetermined number of times.
(9)
The image processing device according to any one of (1) to (8), wherein
the image accumulation section stores the input image and the image importance in association with each other.
(10)
The image processing device according to any one of (1) to (9), further comprising
a display device that displays the image importance.
(11)
The image processing device according to (10), wherein
the display device displays the input image and the image importance.
(12)
The image processing device according to (11), wherein
the display device displays the input image with the image importance being superimposed thereon.
(13)
The image processing device according to any one of (10) to (12), wherein
the display device displays an image indicating that the image importance exceeds a predetermined threshold.
(14)
The image processing device according to (13), wherein
the display device changes a display mode of the image according to the image importance.
(15)
The image processing device according to (13) or (14), wherein
the display device displays the input image with the image being superimposed thereon.
(16)
The image processing device according to (10), wherein
the display device displays the input image, the image importance, and an image indicating that the image importance exceeds a predetermined threshold.
(17)
The image processing device according to (16), wherein
the display device changes a display mode of the image according to the image importance.
(18)
The image processing device according to (16) or (17), wherein
the display device displays the input image with the image importance and the image being superimposed thereon.
(19)
An image processing method comprising:
extracting an intermediate feature amount related to machine learning from an input image that is an image inside a body;
calculating image importance of the input image on a basis of the intermediate feature amount; and
storing the input image on a basis of the image importance.
(20)
A computer-readable recording medium recording a program for causing a computer to execute:
extracting an intermediate feature amount related to machine learning from an input image that is an image inside a body;
calculating image importance of the input image on a basis of the intermediate feature amount; and
storing the input image on a basis of the image importance.
(21)
An image processing method including: performing image processing by the image processing device according to any one of (1) to (18).
(22)
A computer-readable recording medium that records a program for causing a computer to execute a step included in the image processing method according to (21).
(23)
An image processing system including the image processing device according to any one of (1) to (18).

### REFERENCE SIGNS LIST

10 IMAGE PROCESSING SYSTEM
20 ENDOSCOPE
21 RGB CAMERA
30 LEARNING DEVICE
31 INPUT/OUTPUT SECTION
32 LEARNING SECTION
32a FEATURE AMOUNT EXTRACTION SECTION
32b UPDATING SECTION
33 CONTROL SECTION
40 IMAGE PROCESSING DEVICE
41 INPUT/OUTPUT SECTION
42 RECOGNITION SECTION
42a FEATURE AMOUNT EXTRACTION SECTION
42b IMPORTANCE CALCULATION SECTION
42c IMAGE ACCUMULATION SECTION
43 CONTROL SECTION
50 STORAGE DEVICE
60 DISPLAY DEVICE

1. An image processing device comprising:
a feature amount extraction section that extracts an intermediate feature amount related to machine learning from an input image that is an image inside a body;
an importance calculation section that calculates image importance of the input image on a basis of the intermediate feature amount; and
an image accumulation section that stores the input image on a basis of the image importance.

2. The image processing device according to claim 1, wherein
the importance calculation section calculates the image importance on a basis of a difference between the intermediate feature amount of the image inside the body in a first environment and the intermediate feature amount of the input image in a second environment different from the first environment.

3. The image processing device according to claim 2, wherein
the importance calculation section converts the difference by a predetermined conversion formula and calculates the image importance.

4. The image processing device according to claim 2, wherein
the first environment is a first hospital, and
the second environment is a second hospital different from the first hospital.

5. The image processing device according to claim 1, wherein
the image accumulation section stores the input image in a case where the image importance exceeds a predetermined threshold.

6. The image processing device according to claim 5, wherein
the image accumulation section changes the predetermined threshold at update timing of a learned model.

7. The image processing device according to claim 5, wherein
the image accumulation section changes the predetermined threshold according to a number of times of update of a learned model.

8. The image processing device according to claim 7, wherein
the image accumulation section decreases the predetermined threshold at timing at which the number of times of update becomes a predetermined number of times.

9. The image processing device according to claim 1, wherein
the image accumulation section stores the input image and the image importance in association with each other.

10. The image processing device according to claim 1, further comprising
a display device that displays the image importance.

11. The image processing device according to claim 10, wherein
the display device displays the input image and the image importance.

12. The image processing device according to claim 11, wherein
the display device displays the input image with the image importance being superimposed thereon.

13. The image processing device according to claim 10, wherein
the display device displays an image indicating that the image importance exceeds a predetermined threshold.

14. The image processing device according to claim 13, wherein
the display device changes a display mode of the image according to the image importance.

15. The image processing device according to claim 13, wherein
the display device displays the input image with the image being superimposed thereon.

16. The image processing device according to claim 10, wherein
the display device displays the input image, the image importance, and an image indicating that the image importance exceeds a predetermined threshold.

17. The image processing device according to claim 16, wherein
the display device changes a display mode of the image according to the image importance.

18. The image processing device according to claim 16, wherein
the display device displays the input image with the image importance and the image being superimposed thereon.

19. An image processing method comprising:
extracting an intermediate feature amount related to machine learning from an input image that is an image inside a body;
calculating image importance of the input image on a basis of the intermediate feature amount; and
storing the input image on a basis of the image importance.

20. A computer-readable recording medium recording a program for causing a computer to execute:
extracting an intermediate feature amount related to machine learning from an input image that is an image inside a body;
calculating image importance of the input image on a basis of the intermediate feature amount; and
storing the input image on a basis of the image importance.
